(19) United States
(12) Patent Application Publication
(10) Pub. No.: US 2010/0300448 A1
(43) Pub. Date: Dec. 2, 2010

(54) Tracheostomy Tube

(76) Inventors: Michael A. Kenowski, Alpharetta, GA (US); James F. Schumacher, Cumming, GA (US)

Correspondence Address: KIMBERLY-CLARK WORLDWIDE, INC.;Tara Pohlkotte, 2300 Winchester Rd., NEENAH, WI, 54956, US

(21) Appl. No.: 12/473,780

(22) Filed: May 28, 2009

(51) Int. Cl. A61M 16/04

(52) U.S. Cl. 128/207.14

(57) ABSTRACT

There is provided an improved tracheostomy tube having a distal section that over-molds a proximal section. The distal section is desirably made from a more flexible polymer than the proximal section to reduce trauma should the distal section contact the tracheal wall. The insertion of the tracheal tube in the trachea results in large forces being exerted on the tube. Forces acting on the tube after installation can also be rather large. Over-molding of the proximal section by the distal section results in a stronger bond than if the parts are butt-welded. The sections are desirably made from the same polymer and are desirably substantially transparent or clear to visible light so that cameras or other observation means may be used to inspect the tracheal walls through the tracheostomy tube without removing it from the patient.

A tracheostomy procedure involves making a small horizontal incision in the skin of the neck to grant access to the trachea. Because of the uniquely flexible and elastic nature of the trachea, it has been found that healing is much faster if only a small hole is made in the tracheal wall and the hole dilated, rather than cutting the tracheal wall. After the trachea has been dilated, a tracheostomy or “trach” tube is inserted through the stoma. The trach tube includes a balloon or cuff that encircles the tube shaft near its distal end and which is inflated to block the balance of the trachea and direct the ventilating air downward into the lungs. The proximal end of the trach tube is connected to a mechanical ventilator that supplies air through a relatively large central lumen.

The amount of force needed to insert a trach tube into the trachea can cause the tubes to kink and collapse. Great care is needed to avoid this problem, lengthening the time necessary to perform this procedure. In addition, the placement of a trach tube in the trachea is a relatively traumatic procedure in which the tube will be subjected to a multitude of forces such as shear and bending of the tube shaft. Once the tube is placed, it will be subject to additional forces such as axial forces on the vent connector. Known methods of manufacturing include assembly of pre-molded or pre-extruded components as shown in FIG. 1. This method requires bonding, either thermally or with a solvent glue, of numerous components. (Greater discussion of these methods may be found, for example, in U.S. patent application Ser. No. 12/163,173). There are risks associated with these bonded joints and pre-molded internal lumens; the vent connector 2 may break from the proximal section 3, the proximal section 3 may break from the distal section 4, there may be air leakage around a pre-molded and bonded inflation line 6 and groove 8, and there may be leakage out of a glued balloon cuff 10. Other problems caused by the force needed for installation of the tube or caused by the forces generated during usage may also result in failure of a trach tube assembled from individual parts in such a way.

There remains a need for a device that can more quickly and safely allow for the successful placement of a tracheostomy tube and that minimizes the use of thermally or solvent bonded joints and the creation of injection molded internal lumens in order to minimize tube failure.

### SUMMARY

There is provided a novel tracheostomy tube that largely overcomes the problem of trach tube failure due to breakage of connections of pre-molded and pre-extruded parts. In addition, the tube may have a variable flexibility such that the distal end is relatively more flexible than the proximal end, which is advantageous in relation to the trauma that may occur should the distal end of the tube contact the far (posterior) wall of the trachea. The tube may be relatively less flexible at its proximal portion where the greatest amount of force is generally applied during a tracheostomy procedure and after placement by the tracheal rings.

This disclosure also contemplates a new process for the manufacture of a tracheostomy tube that significantly reduces the risk of separation of assembled components during initial placement and/or functional use.

The tube shaft, vent connector, flange, and balloon cuff are desirably made of the same class of materials, e.g.; thermoplastic polyurethane elastomers (TPU) or other thermoplastic elastomers (TPE), which permits over-molding of injection molded components and thermal bonding of the balloon cuff, thereby desirably eliminating the use of glues and solvents for bonding. Importantly, the tube shaft and vent connector can be injection molded as one component, thus eliminating the failure mode wherein the vent connector breaks off from the tube shaft.

The novel process also provides the ability to mold an inner lumen or lumens within the wall of curved tube shaft, eliminating the need for bonding small diameter tubing within a groove along the shaft to serve as an inflation line for the balloon or for a secretion management line. The potential for air leaks from an enclosed lumen is significantly lower than that of a bonded inflation line. Other inherent advantages may include: 1) An internal lumen along the shaft provides a good surface for balloon bonding and allows for the balloon to be thermally bonded to the shaft without interference from a protruding, solvent bonded inflation line. 2) During the fabrication of the internal lumen, an air exit port for balloon inflation may be created at or near the distal end of the tube, eliminating the need to create an air exit port as a secondary operation as is required in the procedure described in the Background above.

### BRIEF DESCRIPTION OF THE DRAWINGS

FIG. 1A is an exploded view of the major components of the prior art tracheostomy tube.

FIG. 1B is an exploded view of the auxiliary components of the prior art tracheostomy tube.

FIG. 2A is a drawing of the pre-molded proximal section of the disclosed tracheostomy tube.

FIG. 2B is a drawing of the cross-section of the proximal section of the disclosed tracheostomy tube at A-A.

FIG. 3A is a drawing of the pre-molded proximal section of the disclosed tracheostomy tube with a disposable core installed.

FIG. 3B is a drawing of the cross-section of the tube of FIG. 3A at B-B.

FIG. 4A is a drawing of the pre-molded proximal section of the disclosed tracheostomy tube with a disposable core installed and an over-molded distal section.

FIG. 4B is a drawing of the cross-section of the tube of FIG. 4A at C-C.

FIG. 5A is a drawing of the disclosed tracheostomy tube after removal of the disposable core and creation of the access to the internal inflation lumen.

FIG. 5B is a drawing of the cross-section of the tube of FIG. 5A at D-D.

FIG. 6 is a drawing of a balloon for a tracheostomy tube as described in U.S. Pat. No. 6,612,305.

FIG. 7 is a drawing of a balloon for a tracheostomy tube as described in U.S. application 60/994,664.

### DETAILED DESCRIPTION

Tracheostomy is a lifesaving procedure to allow a patient to be ventilated directly through the trachea. Tracheostomy is also believed by many to prevent or delay the onset of ventilator acquired pneumonia (VAP).

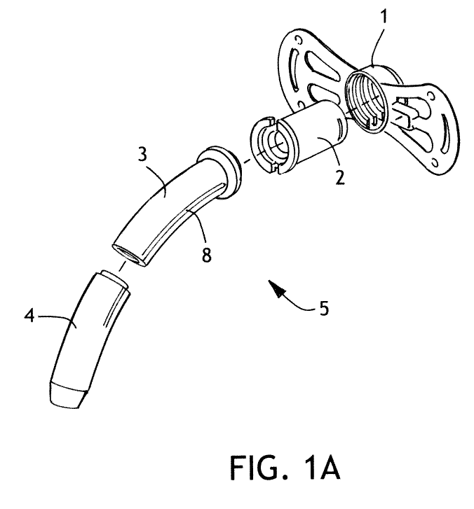

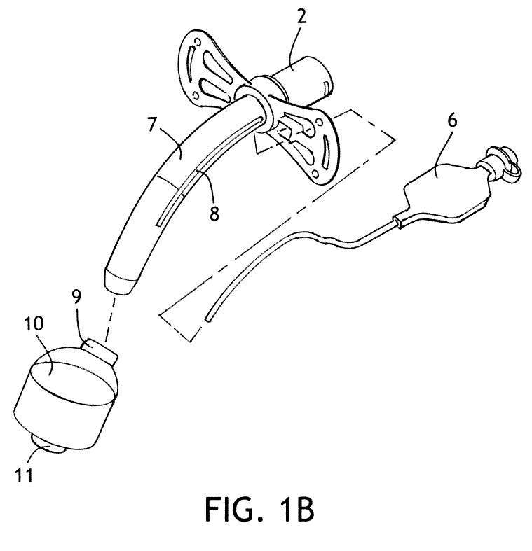

An example of a multi-component, prior art tracheostomy tube is shown in FIGS. 1A and 1B. The tube 5 has a flange 1 on or near the proximal end with a vent connector 2 for connection to the ventilator (not shown). After insertion of the tube 5 in the trachea, the flange 1 will rest against the outside of the throat. The proximal section 3 is bonded on its distal end to the distal section 4 to create the shaft 7. This end-to-end bonding is colloquially known as “butt-welding”. A groove 8 that has been molded into the proximal section 3 and distal section 4 is used to contain an inflation means 6. A balloon 10 is bonded to the shaft 7 near the distal end of the shaft 7 at the balloon's collar ends 9, 11. The upper portion; approximately one third to two thirds of the shaft 7 of the tube 5, extending from below (distal to) the flange 1 in the distal direction, is the area of highest stress when a trach tube is inserted. This high stress area makes the butt-welded parts particularly vulnerable to failure.

The disclosed tracheostomy tube is produced using an over-molding process in order to avoid the failures that may occur in butt-welded tubes. In over-molding applications, additional polymeric material is injection molded around, over, under, or through a substrate material to complete the final part. This injection can be done with a multi-shot process or by insert molding. In insert molding, a substrate must be taken out of the production tool and placed into a different core and cavity to create the volume for the over-mold material. The melt temperature range of the over-mold resin, in general, should be in the same range as the substrate, to enhance bonding. If the melt temperature of the over-mold is too low to melt the surface of the substrate, the bond can be weak. However, if the melt temperature is too high, the substrate might soften and distort. In extreme cases, the over-mold can penetrate the substrate. Choosing compatible materials is critical to ensuring a good bond. In general, compatible materials are of similar chemistry or contain compatible blended components, however, when the substrate and over-mold materials are incompatible, a mechanical interlock can replace the chemical bond. Common problems encountered with over-molding are inadequate chemical or mechanical bonding between the polymers, incomplete filling of one or more components, and flashing of one or more components.

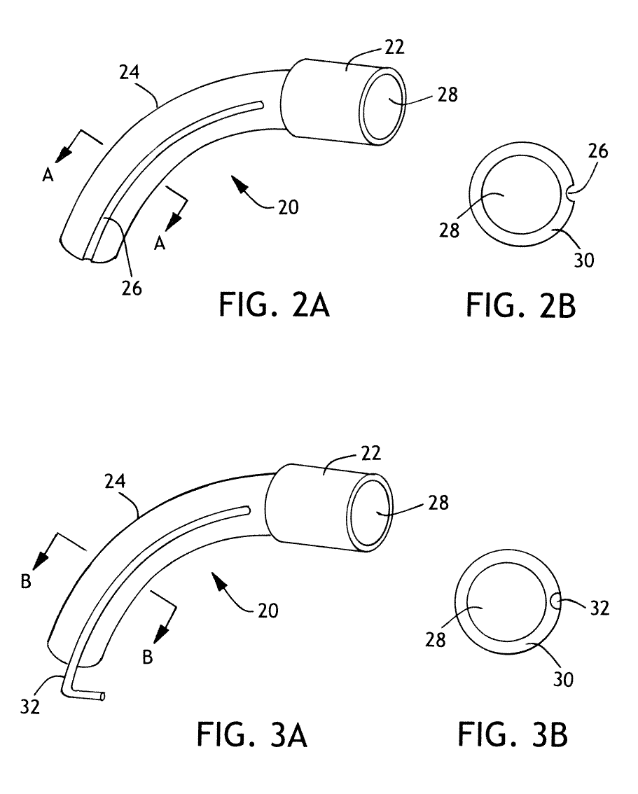

FIG. 2A depicts the proximal portion 20 of an embodiment of the disclosed trach tube. The tube has a central lumen 28. The proximal portion 20 includes a vent connector 22 section and a proximal section 24 that are desirably molded or extruded as a single part. The proximal section 24 includes a small channel or groove 26 that will be used as a balloon inflation line upon completion. The cross-sectional view shown in FIG. 2B across line A-A shows the central lumen 28 of the tube as well as the groove 26 in the tube wall 30.

FIG. 3A again depicts the proximal portion 20 of the embodiment of the disclosed trach tube, including the vent connector 22 and proximal section 24. At this stage, a disposable core 32 has been inserted into the groove 26 on the proximal section 24. The cross-sectional view shown in FIG. 3B across line B-B shows the central lumen 28 of the tube as well as the core 32 in the groove 26 in the wall 30. The tube and core as shown in FIG. 3A are inserted into a mold having the proper desired final dimensions of the part, and additional polymer is injected to over-mold the desired part.

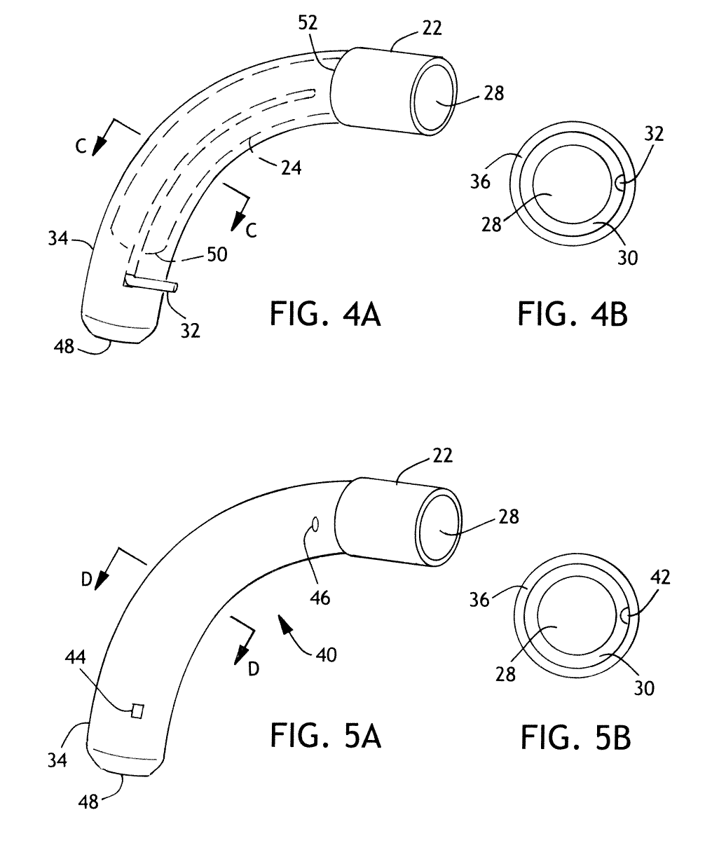

FIG. 4A depicts the proximal portion 20 and disposable core 32 as dashed lines, after being over-molded to create the distal section 34. In the embodiment of FIG. 4A, the distal section 34 extends proximally over the proximal section 24 up to the vent connector 22. This extent of over-molding is desirable, though not required. The distal section 34 may extend toward the vent connector 22 as far as desired by the manufacturer for a particular reason, though less than complete over-molding would likely make for a weaker bond between the distal section 34 and the proximal section 24. The cross-sectional view shown in FIG. 4B across line C-C shows the central lumen 28 of the tube, the core 32 in the groove 26 in the wall 30, and the wall 36 of the distal section 34 where it overlaps the wall 30 of the proximal section 24.

FIG. 5A shows the completed tube shaft 40 having the vent connector 22 and the distal section 34 over-molding the proximal section 24 (not visible). The disposable core 32 has been removed, desirably by sliding it out distally, to create the internal inflation lumen 42. Removal of the disposable core 32 leaves a distal opening 44 on the distal end of the internal inflation lumen 42 for access to the interior of the balloon. The proximal opening 46 to the inflation lumen 42 may be made by skiving (cutting) an access for a tubing line to be connected. In an alternate embodiment the disposable core 32 may have a slight bend upward (away from the tube) on the proximal end so that the disposable core 32 creates the proximal opening 46. The cross-sectional view shown in FIG. 5B across line D-D shows the central lumen 28 of the tube, the inflation lumen 42 in the wall 30, and the wall 36 of the distal section 34 where it overlaps the wall 30 of the proximal section 24.

The disposable core may be, for example, a high temperature thermoplastic that is unaffected by the temperatures at which the polymer is injection molded to produce the distal section 34. Such materials include polyetherimide (PEI), polyetheretherketone (PEEK), polytetrafluoroethylene (PTFE), and polyamideimide (PAI). Other materials include flexible metal wires such as nickel titanium (Nitinol), stainless steel, and aluminum.

In still another alternative embodiment, the disposable core may be dispensed with entirely and the prior art (see FIG. 1B) method described above may be used for the placement of the inflation line. More particularly, a groove may be molded into the over-molded distal section where an inflation line may then be glued or solvent bonded into place. Alternatively the groove may be skived out to create space for the inflation line. While these embodiments do not provide all of the advantages of the embodiment using the disposable core, it does provide most of the advantages while being more conventional from a manufacturing perspective.

The degree of over-molding of the proximal section by the distal section may vary. Desirably the proximal section includes a vent connector that is not over-molded by the distal section. Turning again to FIG. 4A, the distance (taken along a centerline) from the distal end of 52 the vent connector 22 to the distal end 48 of the distal section 34 may be denoted as “L”, also called the tube length. The length of the distal section 34 that does not over-mold the proximal section 24, i.e. from the distal end 50 of the proximal section 24 to the distal end 48 of the distal section 34, may be denoted as “M”, also called the over-mold-free length. And the length of the proximal section from the distal end 52 of the vent connector 22 to the distal end 50 of the proximal section 24 is denoted “N”, also called the over-molded length. Clearly, M plus N must equal L. Desirably, the over-mold-free length divided by the tube length (M/L) is between 0.25 and 0.75, more desirably between 0.3 and 0.5 and most desirably about 0.35.

As mentioned above, the tube may desirably have a variable flexibility or hardness such that the distal section 34 is relatively more flexible than the proximal section 24. This is believed to help reduce trauma should the distal section 34 contact the tissues of the trachea. The relative hardness of the polymers used to make the sections may be measured by the Shore hardness, a series of scales that is known to those skilled in the art. Hardness is measured using a device called a “durometer”, an instrument specifically developed to measure relative hardness, and is usually performed following ASTM D2240. In the Shore A and D hardness or durometer scales, a higher number indicates a polymer that is harder than a polymer having a lower number within each scale. The Shore A and D scales are used for different types of polymers. Typically the Shore A scale is used for softer, more elastic polymers and the Shore D scale used for stiffer polymers. When comparing the Shore A and Shore D scales, low D values are typically harder than high A values. For example, a 55 D hardness is typically harder than a 90 A shore hardness value. Desirably, the distal section of the disclosed tube may have a Shore hardness between 70 A and 90 A and the proximal section may have a Shore hardness between 55 D and 75 D.

The proximal and distal sections of the tube are desirably made from the same material (though of different hardnesses) as the balloon cuff so that joining the components may be easily accomplished. These materials include thermoplastic polyurethane elastomers, thermoplastic polyolefin elastomers, thermoplastic polyolefin block copolymers, SBS di-block elastomers, SEBS tri-block elastomers, polyvinyl chloride, polyethylene terephthalate and blends and mixtures thereof. A particularly suitable polymer is polyurethane. In one embodiment the proximal section may be made from Dow Chemical's thermoplastic polyurethane elastomer PELLETHANE, type 2363-75D. The distal section may be made from PELLETHANE 2363-80 A and the balloon made from PELLETHANE 2363-90A. In each case the polymer is a grade of polyurethane designated 2363 but the hardness varies as indicated by the last two numbers and the letter.

It is also desirable that the sections be substantially clear or transparent so that a camera or other means of viewing the interior of the trachea using the visible light spectra may be used to investigate the condition of the tracheal wall. By “substantially clear or transparent” is meant that the section is sufficiently transparent that the selected viewing means may see the tracheal wall through the section without the use of x-rays or other non-visible waves. Medical professionals find it desirable to examine the condition of the trachea to check for signs of infection, erosion or other inflammation and the ability to merely insert a device for viewing through the tracheostomy tube is an advantage. The alternative is to insert a device through the patient's mouth, causing great discomfort as well as irritating the vocal cords, or to remove the trach tube to view the trachea directly through the tracheostomy stoma. Since the patients on whom this procedure is used are generally in poor health, avoiding such procedures is desirable. If the tracheostomy tube and balloon are substantially clear or transparent as disclosed herein, the medical professional can simply and relatively safely see the condition of the trachea without removing the tube from the patient. The selection of the proper polymer can produce a tube and balloon combination that are substantially clear or transparent and the Dow Chemical polymers mentioned above are suitable for this purpose.

Once the tube is completed it may be attached to the flange by adhesive or solvent bonding. The flange may also be over-molded if desired, if suitable materials are used. A suitable material is, for example, Dow Chemical's PELLETHANE 2363-80 A.

As mentioned above, the trach tube has a balloon cuff around its circumference on a lower (distal) portion of the tube that serves to block the normal air flow in the trachea so that (assisted) breathing takes place through the trach tube using a ventilator. The cuff is desirably made from a soft, pliable polymer such as polyurethane (PU), polyethylene teraphihalate (PETP), low-density polyethylene (LDPE), polyvinyl chloride (PVC), or elastomeric-based polyolefins. It should be very thin; on the order of 25 microns or less, e.g. 20 microns, 15 microns, 10 microns or even as low as 5 microns in thickness. The cuff should also desirably be a low pressure cuff operating at about 30 mmH₂O or less, such as 25 mmH₂O, 20 mmH₂O, 15 mmH₂O or less. Such a cuff is described in U.S. Pat. No. 6,802,317 which describes a cuff for obturating a patient's trachea as hermetically as possible, comprising a cuffed balloon which blocks the trachea below a patient's glottis, an air tube, the cuffed balloon being attached to the air tube and being sized to be larger than a tracheal diameter when in a fully inflated state and being made of a soft, flexible foil material that forms at least one draped fold in the cuffed balloon when inflated in the patient's trachea, wherein the foil has a wall thickness below or equal to 0.01 mm and the at least one draped fold has a loop found at a dead end of the at least one draped fold, that loop having a small diameter which inhibits a free flow of secretions through the loop of the at least one draped fold. Another description of such a cuff is in U.S. Pat. No. 6,526,977 which teaches a dilator for obturating a patient's trachea as hermetically as possible, comprising a cuffed balloon which blocks the trachea below a patient's glottis, an air tube, the cuffed balloon being attached to the air tube and being sized to be larger than a tracheal diameter when in a fully inflated state and being made of a sufficiently soft, flexible foil material that forms at least one draped fold in the cuffed balloon when fully inflated in the patient's trachea, wherein the at least one draped fold formed has a capillary size which arrests free flow of secretions across the balloon by virtue of capillary forces formed within the fold to prevent aspiration of the secretions and subsequent infections related to secretion aspiration.

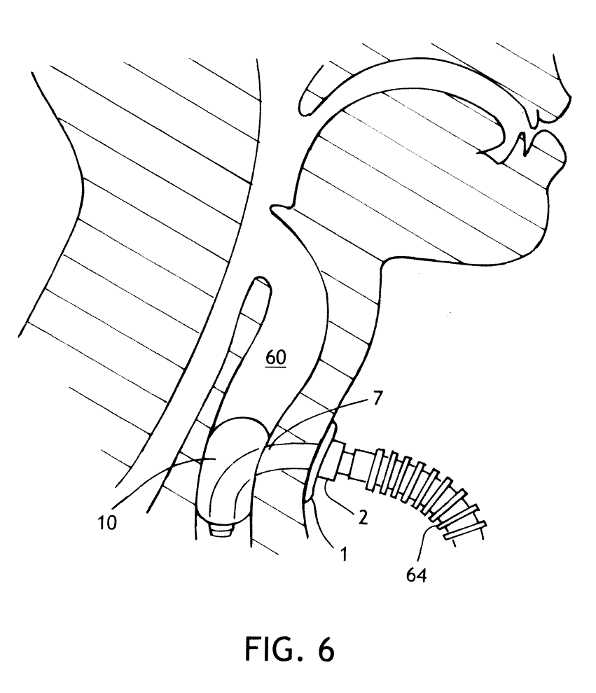

Alternatively, the balloon may be of a shape as described in U.S. patent application 60/994,664, now 12/206,517 or U.S. Pat. No. 6,612,305. In the '305 patent, the balloon 10 expands in the trachea 60 not only around the tube 7, as do the current models, but also cranially to it and to the stoma, sealing the stoma (FIG. 6). FIG. 6 also shows the flange 1, ventilator connector 2 and the ventilator line 64. Sealing of the stoma in the '305 device is achieved by the fact that the proximal point of attachment and the distal point of attachment of the inflatable cuff on the tube are not contiguous or, in other words, are at an angle (α) other than 180 degrees, relative to conventional devices.

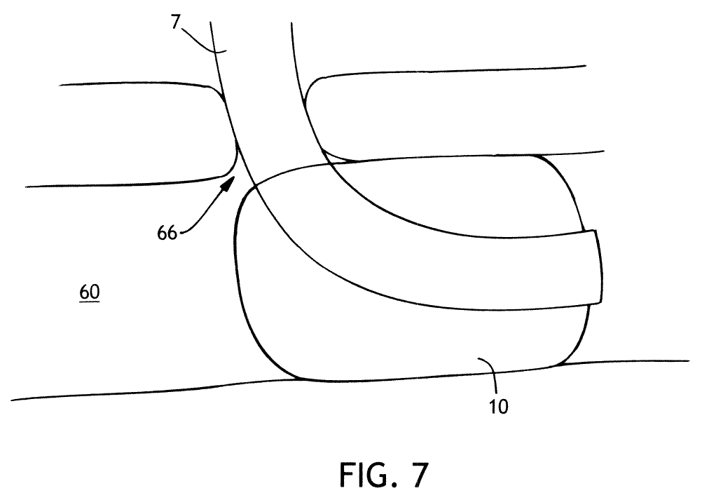

In the '644 application, the balloon 10 has a distal balloon portion substantially centered about and attached to the distal end portion of the tube 7. The balloon also has a proximal balloon portion attached to the bend region of the tube and positioned substantially off-center about the bend region below the proximal plane of the device. Upon inflation, this configuration provides for expansion of the balloon around the distal end portion of the tube and the proximal end portion of the tube below the proximal plane of the device to seal the trachea below the tracheal stoma 66 and avoid sealing the trachea above the tracheal stoma (FIG. 7). Desirably, this configuration of the balloon will allow secretions to exit the stoma.

The tracheostomy tube device may have balloon walls that are non-uniform in thickness. For example, the device may have a first portion of the balloon in which the walls have a thickness of about 20 to 30 micrometers and a second portion of the balloon in which the walls have a thickness of about 5 to about 15 micrometers. Desirably, the first portion of the balloon is the portion of the balloon contacting the upper portion of a cross-sectional region of the tracheal lumen and the second portion of the second balloon is the portion of the balloon contacting the lower portion of the same cross-sectional region of the tracheal lumen.

The inflatable balloon component may include a distal end, a distal attachment zone, a proximal end, a proximal attachment zone, an upper region and a lower region, wherein the upper region has a thickness of from about 15 to about 30 micrometers and the lower region has a thickness of from about 5 to about 15 micrometers.

The balloon component may desirably be formed from thermoplastic polyurethane polymers, thermoplastic polyolefin elastomers, thermoplastic polyolefin block copolymers, SBS di-block elastomers, SEBS tri-block elastomers, polyvinyl chloride, polyethylene terephthalate and blends and mixtures thereof.

The trach tube also may be used with disposable cannulas that are placed within the trach tube from the proximal end. These disposable cannulas are changed regularly so that bacterial growth is kept to a minimum. The cannulas are made from a plastic material such as a polyolefin, polyurethane, nylon, etc and are desirably semi-rigid. Cannulas may be treated with anti-bacterial and/or anti-viral coatings or other active materials to help reduce the growth of harmful organisms.

As will be appreciated by those skilled in the art, changes and variations to the invention are considered to be within the ability of those skilled in the art. Such changes and variations are intended by the inventors to be within the scope of the invention. It is also to be understood that the scope of the present invention is not to be interpreted as limited to the specific embodiments disclosed herein, but only in accordance with the appended claims when read in light of the foregoing disclosure.

What is claimed is:

1. A tracheostomy tube comprising a relatively less flexible proximal section over-molded by a relatively more flexible distal section.

2. The tracheostomy tube of claim 1 wherein said distal section has a Shore hardness between about 70 A and 90 A.

3. The tracheostomy tube of claim 2 wherein said proximal section has a Shore hardness between about 55 D and 75 D.

4. The tracheostomy tube of claim 1 wherein an over-mold-free length divided by a tube length (M/L) is between 0.25 and 0.75.

5. The tracheostomy tube of claim 4 wherein M/L is between 0.3 and 0.5.

6. The tracheostomy tube of claim 1 further comprising a balloon cuff made from a soft, pliable polymer and having a thickness between 5 and 25 microns.

7. The tracheostomy tube of claim 6 wherein said polymer is selected from the group consisting of thermoplastic polyurethane polymers, thermoplastic polyolefin elastomers, thermoplastic polyolefin block copolymers, SBS di-block elastomers, SEBS tri-block elastomers, polyvinyl chloride, polyethylene terephthalate and blends and mixtures thereof.

8. The tracheostomy tube of claim 6 wherein said 11 wherein said tube is substantially clear or transparent.

9. A method of making a tracheostomy tube comprising the steps of providing a tube proximal section, placing the proximal section in a mold, and injecting a polymer into said mold to over-mold the proximal section.

10. The method of claim 9 wherein injecting said polymer produces a distal section.

11. The method of claim 10 wherein said distal section is relatively more flexible than said proximal section.

12. The method of claim 10 wherein said proximal section and said distal section are substantially clear or transparent.

13. The method of claim 10 wherein said proximal section and said distal section are made of polyurethane.
